(19) United States
(12) Patent Application Publication
(10) Pub. No.: US 2007/0287945 A1
(43) Pub. Date: Dec. 13, 2007

(54) Ingrown toenail treatment method

(76) Inventor: Byung Kwon Cha, Brooklyn Park, MN (US)

Correspondence Address: Byung Kwon Cha, Suite 4, 1401 Silver Lake Road, New Brighton, MN, 55112, US

(21) Appl. No.: 11/448,497

(22) Filed: Jun. 7, 2006

(51) Int. Cl. A61F 5/11

(52) U.S. Cl. 602/31; 128/898

(57) ABSTRACT

A method for treating an ingrown toenail by providing a smooth surface on the lateral edge of the damaged/sharpened nail and isolating the lateral nail bed, i.e., the cuticle, from the sharpened nail, thereby aborting the cycle of mutual damage that occurs when an ingrown toenail causes inflammation on the flesh portion at the side or under the toenail which in turn then further dissolves the nail edge and sharpen it, which in turn then causes further damage and inflammation. By ceasing this cycle, the sufferer of the ingrown toenail feels immediate pain relief while the toenail heals itself by means of isolation of the cuticle from the sharpened portion of the nail.

### PRIOR APPLICATION

This application emanates from a prior Provisional Patent Application filed May 26, 2005.

### FIELD OF THE INVENTION

This invention relates to the field of treatment for the condition of ingrown toenails. In particular, this invention involves a method that a person can use without the assistance of medical personnel.

### BACKGROUND OF THE INVENTION

An ingrown toenail occurs when part of the toenail penetrates the fleshy part of the skin. This often results in an infection. Usually one side of the nail penetrates deep into the toe, making it difficult to see the edge of the nail. The corner or a small spike of nail penetrates the skin, just like a knife. This can result in an infection and the development of proud flesh, i.e., granulation tissue. The toe will then be red, inflamed and painful.

Pain is the main symptom of an ingrown toenail, usually starting out as just minor discomfort.

This may be just the pressure from the side of the nail or it may be because the nail has actually penetrated the skin down the side of the nail. The toe is not necessarily infected, but an infection can develop after the nail penetrates the skin and becomes ingrown. The infection can then spread, making the toe red and inflamed. A collection of pus may also develop.

Poor cutting of the nail is the most commonly held belief as to the cause of ingrown toenails, but other factors such as poorly fitted shoes, infections and other traumas are also believed to cause ingrown toenails. The cornerstone of self-treatment and prevention of ingrown toenails involves the cutting of the nail straight across the width of the nail, allowing the corners to protrude, ostensibly so that they do not penetrate the skin. The toenails are typically cut straight across without tapering the corners. However, this is not always easy, especially if the nail is very curved down the side.

It is a myth that a “V” should be cut in the end of the nail to treat an ingrown toenail. The apparent reasoning behind this is that if you cut a “V” in the nail, the edge of the nail will grow together as the nail grows out. This does not actually occur, as the growing area at the base of the toe rather than at the end determines the shape of the nail.

Existing efforts to treat ingrown toenails involve the restoration of the shape of the toenail. However, those efforts have not been successful in all cases because the misshaped toenail is not the real cause of the pain. The infection and cycle of pain and damage that occurs once the condition has started has little to do with the shape of the nail. The ingrown toenail starts with damage to the lateral nail bed, i.e., the cuticle that often develops into a sharpened edge. The damaged cuticle then produces an inflammatory reaction in the toe. In turn, the matter produced by the inflammation further dissolves the lateral side of the nail, making the edge even sharper, thereby producing further cutting into the skin, further damaging the cuticle and causing further infection and inflammation. Unless there is an interruption in this cycle of mutual damage, the problem will steadily worsen.

Prior art in the field addresses the theory that reshaping the nail will solve the problem. Vironda in U.S. Pat. No. 3,981,298 describes a system wherein a liquid material is applied to a nail in a limited area, which hardens and then serves as a form-retaining support. This invention involves the application of a strip that spreads along the entire width of the nail. However, this invention only restores the shape of the nail and does not address the cycle of inflammation and nail damage that is occurring at the site of the inflammation. Steele in U.S. Pat. No. 5,862,811 defines a system that involves an adhesive and solvent that fills cracks in nails with polystyrene pellets. Like the Vironda invention, the Steele invention does not seek to isolate the problem at the damaged point of the nail, but merely seeks to reshape the nail in hopes that it will grow out of the embedded fleshy portion of the toe. Similarly, Machida in U.S. Pat. No. 6,095,995 teaches of a plate-like member that is made from a shape-memorizing alloy or resin material that is designed to match the curvature of the nail at room temperature which then deforms into a memorized flat shape when heated.

As with the previous inventions, Machida seeks only to reshape the nail. Accordingly, there exists a need for a system that isolates the sharpened edge of the nail and provides a smooth surface on the lateral edge of the nail, thereby reducing the cycle of inflammation, infection, damage and pain.

### SUMMARY OF THE INVENTION

In the preferred embodiment of the invention, a method is described in the following steps. First the toe with the ingrown toenail is soaked in antiseptic solution-mixed warm water to soften it, for approximately ten minutes. The toe is then cleaned with soap and then completely rinsed with clean water. The toe is then dried with a towel. The toe is then disinfected again on the side of the damaged portion as well as underneath it. This is accomplished through the application of an antiseptic solution that is absorbed into a small piece of cotton ball. The cotton ball is guided with a toothpick to the affected area.

Using a finger, the damaged flesh part at the side of the nail bed is pushed outward and a U-shaped protecting gutter that is made out of soft plastic material is gently inserted to the lateral edge of the damaged toenail, and then the finger that was pushing the side of the nail bed outward is released. A non-toxic liquid bonding agent is then applied to cover the entire affected area, both underneath the toenail and on the side of the toenail. The bonding agent then must be dried. This can be accomplished through ambient temperature or can be accelerated through the use of a hair dryer, or other mechanical heating means. Once the bonding agent has dried, the pain should immediately cease. The outer part of the protecting gutter that is sticking out from the treated toenail area is carefully trimmed off. The toenail should then be checked daily and washed with soap. The antiseptic solution should also be re-applied daily. If the bond between the nail and the side of the nail bed becomes separated, the bonding agent should be re-applied. Once the portion of the nail containing the protecting gutter and bonding agent grows to a sufficient length, the nail can then be clipped and discarded.

### OBJECT OF THE INVENTION

The principal object of the invention is to provide a smooth surface on the lateral edge of the damaged/sharpened nail and isolating the lateral nail bed, i.e., the cuticle, from the sharpened nail, effectively stopping the cycle of mutual damage occurring between the sharpened edge of the nail and inflamed fleshy portion of the toe.

### BRIEF DESCRIPTION OF THE DRAWINGS

FIG. 1 illustrates a top view of a toe affected with an ingrown toenail.

FIG. 2 illustrates a view of an affected toe soaked in antiseptic solution-mixed warm water to soften it.

FIG. 3 illustrates a top view of an affected toe being treated with an antiseptic solution using a cotton ball and a toothpick.

FIG. 4 illustrates the shape of a protecting gutter, and shows how the protecting gutter covers up the lateral edge of the damaged and sharpened nail.

FIG. 5 illustrates a top view of an affected toe with a finger pushing the affected flesh portion of the toe outward and two fingers of the other hand insert the protecting gutter to the lateral edge of the damaged toenail

FIG. 6 illustrates a top view of an affected toe with the protecting gutter inserted and the liquid bonding agent applied to the affected area.

FIG. 7 briefly illustrates a front view of a normal toe and toenail

FIG. 8 briefly illustrates a front view of a toe with an ingrown toenail

FIG. 9 briefly illustrates a front view of a toe with an ingrown toenail that has been treated with protecting gutter and bonding agent.

### DETAILED DESCRIPTION OF AN ENABLING AND PREFERRED EMBODIMENT

For a better understanding of the invention, we turn now to the drawings. FIG. 1 offers a visual understanding of how an ingrown toenail appears to the eye. The toe 1 has a nail 2 that has a portion 3 that is cutting into the flesh of the skin and creating an infection. To treat this condition, first the toe 1 is soaked in antiseptic solution-mixed warm water to soften it, cleaned with soap and water, rinsed and then dried. FIG. 3 shows the toe 1 that being disinfected on the side and the underneath of the ingrown toenail 3 through the application of an antiseptic solution that is absorbed into a small piece of cotton ball 5. The cotton ball 5 is then guided with a toothpick 4 to the affected area 3.

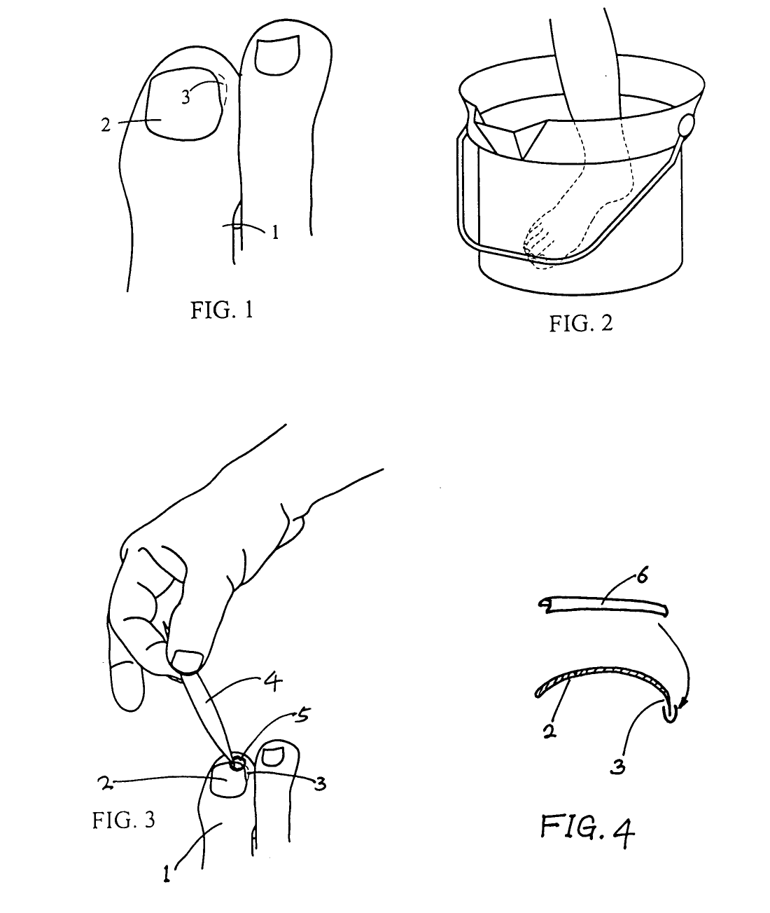

FIG. 4 shows the brief shape of a protecting gutter 6 that is made out of soft plastic material for comfortable feeling and has U-shaped cross section in order to wrap up the damaged edge of ingrown toenail, and shows how the said protecting gutter covers up the lateral edge of the damaged nail.

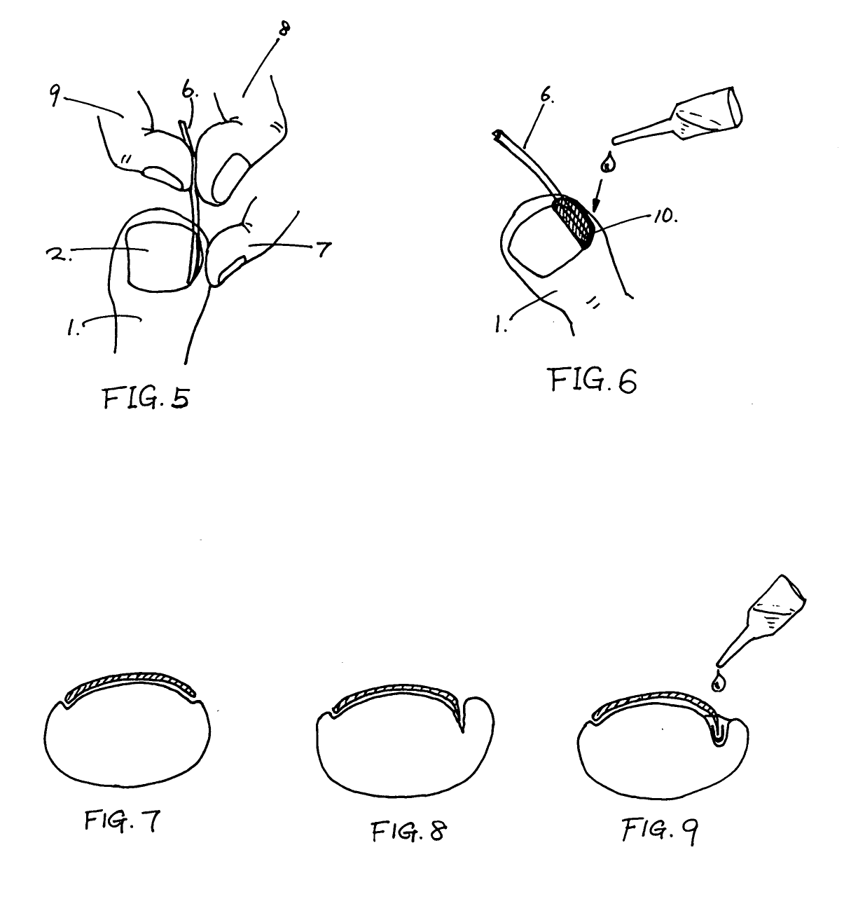

FIG. 5 shows the use of a finger 7 to push the damaged flesh part of the affected toe outward while two fingers 8 and 9 from the other hand gently insert the protecting gutter to the lateral edge of the damaged toenail.

FIG. 6 shows where the liquid bonding agent 10 has been applied to the damaged area.

The illustrations and examples provided herein are for explanatory purposes and are not intended to limit the scope of the appended claims, as those skilled in the art will make modifications to the invention for particular uses.

I claim:

1. A method for treating an ingrown toenail comprising
soaking a toe suffering from an ingrown toenail in antiseptic solution-mixed warm water for a sufficient amount of time to soften said toe;
washing said toe with a soap and clean water;
drying said toe after said washing;
soaking an antiseptic solution onto an absorbent means;
applying said antiseptic solution-soaked absorbent means through the use of a guiding means to said toe, applying said antiseptic solution over the entirety of said toenail;
gently pushing the damaged flesh part at the side of the said ingrown toenail outward with a pushing means;
inserting a protecting gutter to the side of said ingrown toenail to cover the damaged and sharpened edge of the said ingrown toenail;
applying a bonding means to cover the entire affected area of said toenail, coating the underneath and side of said ingrown toenail;
allowing said bonding means to dry;
trimming off the outer part of the said protecting gutter that is sticking out from the treated toenail area;
continuing to wash said toenail daily and applying said antiseptic solution until said toenail has grown to a sufficient length to be clipped off and discarded;
re-applying said bonding agent as needed until the said toenail grows sufficiently so that healthy new toenail replaces the said ingrown toenail;

2. A method according to claim 1 wherein said toe is soaked in antiseptic solution-mixed warm water for ten minutes at the first step.

3. A method according to claim 1 wherein said toe is washed with soap and clean water.

4. A method according to claim 1 wherein said toe is dried using a towel.

5. A method according to claim 1 wherein said absorbent means is a cotton ball.

6. A method according to claim 1 wherein said guiding means is a toothpick.

7. A method according to claim 1 wherein said pushing means is a finger.

8. A method according to claim 1 wherein said protecting gutter is a small piece of plastic stick that has U-shaped cross section and is made out of soft plastic material.

9. A method according to claim 1 wherein the said bonding means is any bonding agent that can bond onto the human toenail. A non-toxic fast drying liquid bonding agent is preferred for the said bonding means, however it does not limit usage of any other type of bonding agent.

10. A method according to claim 1 wherein said bonding agent is dried at room temperature.
